(19) United States
(12) Patent Application Publication
(10) Pub. No.: US 2014/0163505 A1
(43) Pub. Date: Jun. 12, 2014

(54) Wound Dressing

(71) Applicant: Leslie Clemens, Azle, TX (US)

(72) Inventor: Leslie Clemens, Azle, TX (US)

(73) Assignee: SESSIONS PHARMACEUTICALS INC., Fort Worth, TX (US)

(21) Appl. No.: 14/009,494

(22) PCT Filed: Apr. 4, 2012
(86) PCT No.: PCT/US12/32162

§ 371 Date: Dec. 6, 2013

Related U.S. Application Data
(60) Provisional application No. 61/471,610, filed on Apr. 4, 2011.

(51) Int. Cl. A61F 13/00

(52) U.S. Cl. CPC A61F 13/00042
USPC 604/369

(57) ABSTRACT

A foam-based wound dressing for covering a wound on an extremity. The wound dressing includes a composite body having two distinct layers joined along a portion of their perimeters to form a least one open end. Each layer includes a hydrophilic foam matrix attached to a urethane film.

### CROSS-REFERENCE TO RELATED APPLICATION

This patent application claims the benefit of U.S. Provisional Patent Application No. 61/471,610, filed Apr. 4, 2011, which is incorporated by reference in its entirety herein.

### BACKGROUND

In the management of wounds, such as wounds to extremities, it is desirable to cover and protect such wounds while promoting gradual closure. It is also desirable to keep such wounds free from external contamination, or slough or debris from the wound. It is further desirable to keep such wounds sterile. It is further desirable to avoid substantial accumulation of wound exudates at the wound situs, such as blood, pus, and other wound fluids, since the presence of accumulated exudates may promote the growth of bacteria or other microorganisms, which delay the healing process.

Wound dressings comprising bandages with cotton absorption pads or gauze have long been used to treat such wounds. After application over the wound, these dressings may absorb the fluid exudate and are subsequently removed and replaced with a new dressing in accordance with known wound care protocols. These dressings also may include antiseptics and/or anti-bacterial agents such as silver-based compositions and the like.

### BRIEF SUMMARY

The present invention provides a foam-based wound dressing incorporating a sleeve for covering an extremity such as an arm, leg, hand, foot, finger, toe, or penis.

In accordance with one aspect, the present invention provides a wound dressing including a composite body of suitable length adapted for covering a wound on an extremity. The body includes two distinct layers joined along a portion of their perimeters to form either a closed end and an open end or two open ends. Each layer can include a hydrophilic foam matrix attached to a urethane film.

In accordance with another aspect, the present invention provides a method for treating a wound on an extremity. The method includes providing a wound dressing having a composite body of suitable length adapted to foam a sleeve for covering a wound on an extremity. The wound dressing includes two distinct layers joined along a portion of their perimeters. The wound dressing has an open end and a closed end. The closed end is disposed opposite the open end and can be curved. Each layer can include a hydrophilic foam matrix attached to a urethane film. The closed end can be folded into the sleeve to provide a starting point for inserting a tip of an extremity to begin rolling the wound dressing over the extremity. The wound dressing is retained over the wound for a time sufficient to allow at least partial healing of the wound while wicking fluid away from the wound.

In accordance with a further aspect, the present invention provides a method for treating a wound on an extremity. The method includes providing a wound dressing having a composite body of predetermined length adapted to form a sleeve for covering a wound on an extremity. The wound dressing includes two distinct layers joined along a portion of their perimeters. The wound dressing can have two open ends. Each layer can include a hydrophilic foam matrix attached to a urethane film. The wound dressing is retained over the wound for a time sufficient to allow at least partial healing of the wound while wicking fluid away from the wound.

While the invention will be described in connection with the illustrated embodiments, it is understood that the invention is not intended to be so limited. On the contrary, it is intended to cover all alternatives, modifications, and equivalents as may be included within the spirit and scope of the invention as defined by the appended claims.

### BRIEF DESCRIPTION OF THE DRAWINGS

FIG. 1 is a plan view of an embodiment of a wound dressing for an extremity;

FIG. 2 is an exploded perspective view of the wound dressing of FIG. 1 and a dressing insert;

FIG. 3 is a perspective view of the wound dressing and dressing insert of FIG. 2;

FIG. 4 is a plan view of the wound dressing and dressing insert of FIG. 2;

FIG. 5 is a perspective view of the dressing insert shown in FIG. 2;

FIG. 6 is another perspective view of the dressing insert of FIG. 5 with a tab folded;

FIGS. 7-12 show the process of applying the wound dressing of FIG. 1 to an extremity;

FIG. 13 is a perspective view of the wound dressing of FIG. 1 in a partially unrolled configuration;

FIG. 14 is a perspective view of the wound dressing of FIG. 1 disposed over a finger;

FIG. 15 is an enlarged fragmentary section view of the wound dressing of FIG. 1 taken through line 15-15

FIG. 16 is a simplified view of the cutting and welding operation to form a wound dressing;

FIG. 17 is a perspective view of a wound dressing on a finger;

FIG. 18 is a perspective view of a wound dressing on a hand;

FIG. 19 is a perspective view of a wound dressing on a limb;

FIG. 20 is a perspective view of a wound dressing on an arm;

FIG. 21 is a perspective view of a wound dressing on a leg; and

FIG. 22 is a perspective view of a wound dressing on a foot.

### DETAILED DESCRIPTION

Reference will now be made to the drawings, wherein like elements are designated by like reference numerals throughout the various views.

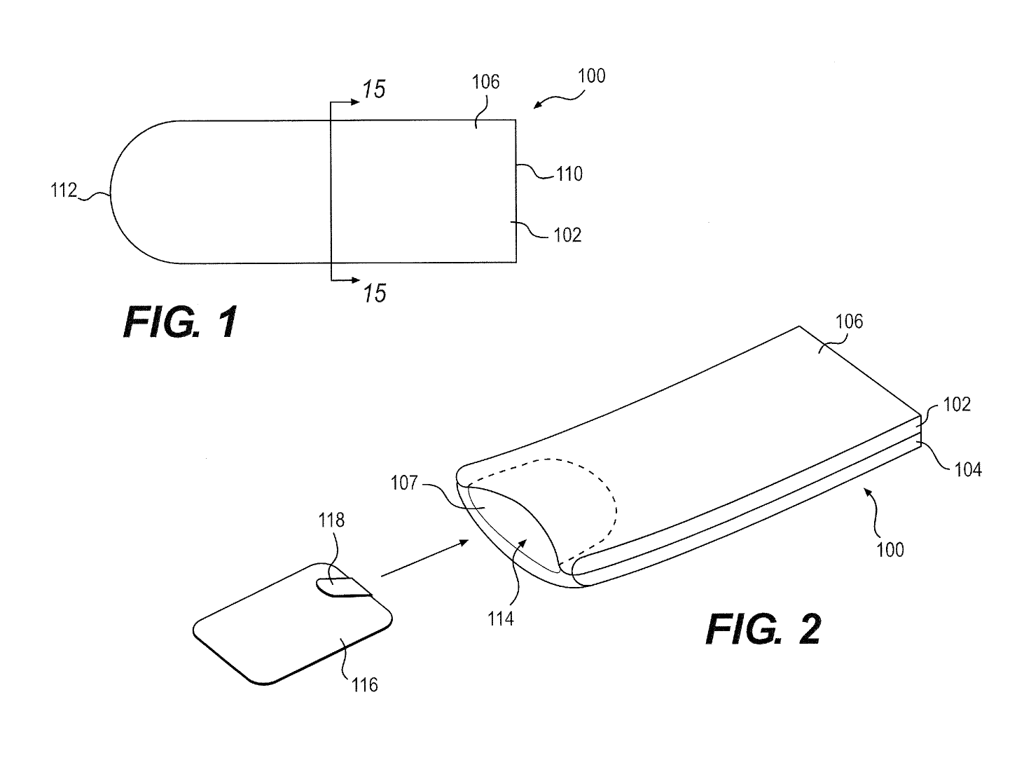

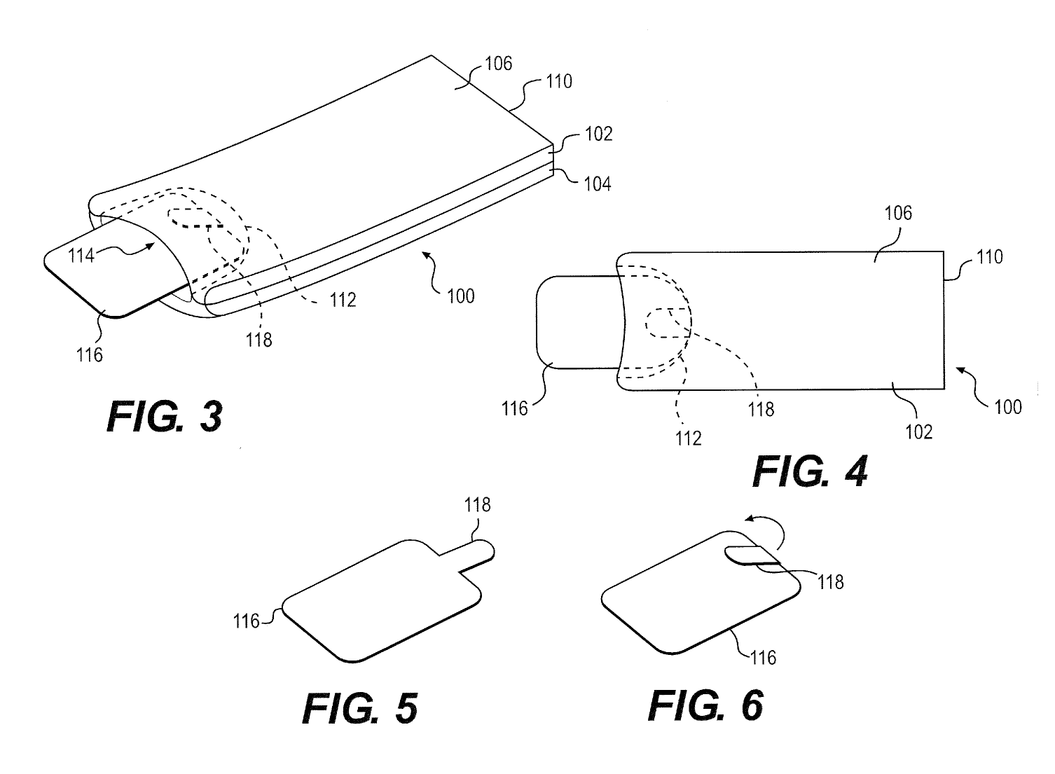

Referring jointly to FIGS. 1-4 and 17, the wound dressing 100 can be formed from a polymer-based foam as will be described further hereinafter. The wound dressing 100 can have a first layer 102 and a second layer 104. Each layer can include the polymer-based foam 106, 107 joined to a urethane film 108, 109. The first layer 102 and the second layer 104 can be joined to one another along a portion of their perimeters to form an elongated sleeve with a pocket formed between the layers 102, 104. The layers 102, 104 can be joined via any suitable method, e.g., via adhesives (e.g., bead or pressure sensitive adhesives), welding (e.g., heat or ultrasonic welding), or stitching, with the unjoined side or sides forming an open end 110. A curved closed end 112 can be disposed opposite the open end 110, with the shape of the curved closed end 112 generally approximating the curvature of an extremity such as a tip of a finger, toe, amputated limb, or penis. In other embodiments, the wound dressing can include a second open end opposing the open end 110.

The wound dressing 100 is intended to fit over and at least partially enclose an extremity. In order to ease the insertion of the wounded finger or toe into the wound dressing 100 without further damaging the wound or causing unnecessary additional pain, the wound dressing 100 may be provided to the user in a configuration permitting the wound dressing 100 to be rolled over itself. Thus, when provided to a user, the foam portion 106, 107 of each layer 102, 104, which is intended to contact the wound, can initially be disposed on the exterior of the wound dressing 100 and then be rolled to form the interior.

To provide the user with an insertion position for an extremity, the curved closed end 112 can be folded to form a dimple 114. Referring to FIGS. 2-6, a dressing insert 116 can be provided to prevent the foam portion 106, 107 of the layers 102, 104 from sticking to one another inside the dimple 114, and to provide a visual indication to a user as to which end the extremity should be inserted. The dressing insert 116 can be generally rectangular with a tab 118 extending from an end. The tab 118 can be folded prior to insertion into the dimple 114. After insertion of the tab 118 into the dimple 114, the tab 118 helps to retain the dressing insert 116 within the dimple 114 until removed be a user. It will be appreciated that the tab can be any suitable shape or size.

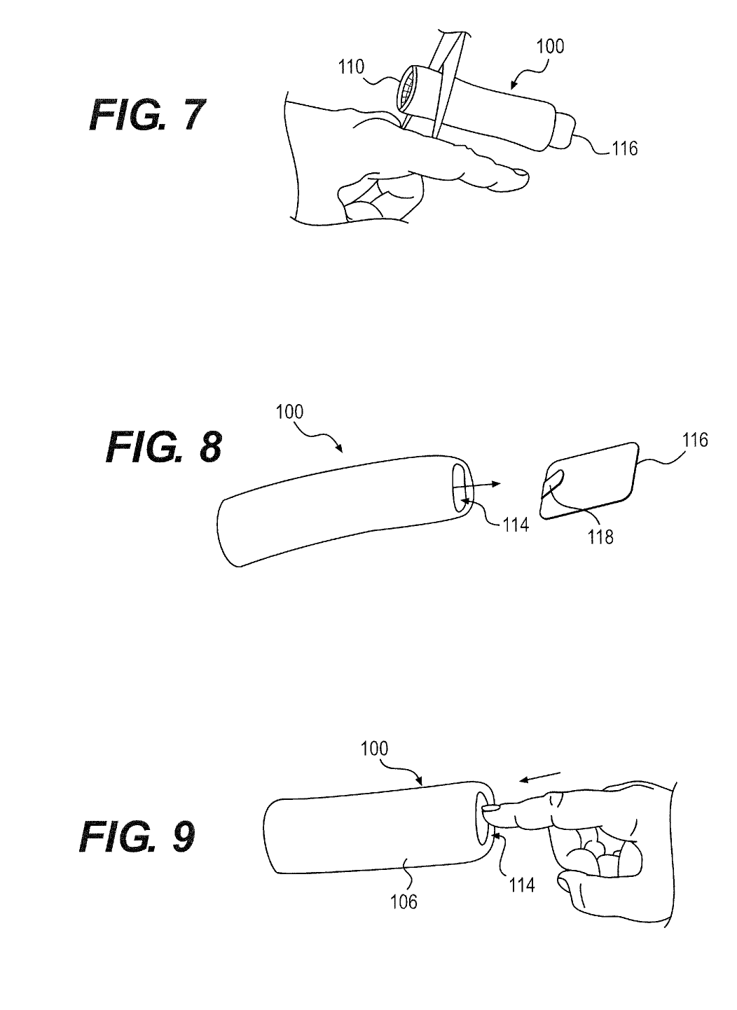

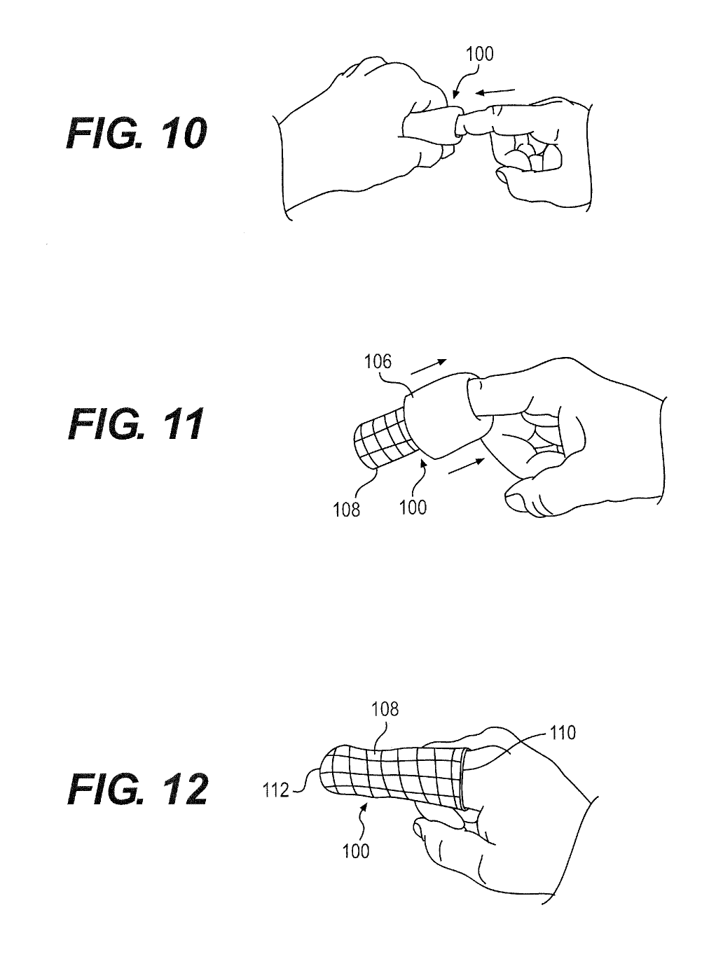

Referring to FIG. 7, in order to apply the wound dressing 100 to a wound on an extremity, such as a finger, a user can trim the wound dressing 100 near the open end 110 to the proper size for the extremity with the wound. As shown in FIG. 8, a user can then remove the dressing insert 116 from the dimple 114. The dressing insert 116 can be discarded. As shown in FIG. 9, a portion of the wounded extremity, such as the tip, is inserted into the dimple 114 as a starting point for covering a portion of the extremity. As shown in FIGS. 10 and 11, the wound dressing 100 can then be gently pushed toward the user to begin rolling the wound dressing 100 over itself such that the foam portions 106, 107 of the first and second layers 102, 104 contact and enclose at least a portion of the extremity. As shown in FIGS. 12 and 14, the user will continue to completely roll the wound dressing over itself until the entire foam portions 106, 107 of the first and second layers 102, 104 are disposed within the sleeve, and the entire film portions 108, 109 of the first and second layers 102, 104 are disposed on the exterior of the sleeve. When fully unrolled over the extremity, the closed end 112 is disposed near an end of the extremity, such as the tip of the finger, and the open end 110 is disposed away from the end of the extremity, such as near the hand. The film portions 108, 109, which are desirably gas-permeable and liquid-impermeable, mitigate water or other contaminants from entering the interior of the sleeve. The film portions also reduce the amount of exudate that can reach the exterior of the wound dressing 100. As shown in FIG. 12, the film portions 108, 109 can include indicia, such as the illustrated grid pattern, which can help to identify the portion of the wound dressing that should be disposed on the exterior and away from the wound.

The wound dressing 100 can be left covering the wound for any suitable number of days. For example, in some embodiments, the wound dressing can be left covering the wound for several days. In other embodiments, the wound dressing can be left covering the wound for as many as seven days. In certain embodiments, the wound dressing should be replaced if exudate is visible outside the boundary of the covered wound. The wound dressing 100 can be used to treat any suitable injury, such as sprains, strains, contusions, abrasions, lacerations, burns, ulcers, and matricectomies.

The wound dressing 100 can be any suitable shape or size. In some embodiments, the wound dressing 100 can be approximately 4″×1.25″, and the weld can be along approximately 9.5 linear inches. Each layer can be approximately 0.125″ thick.

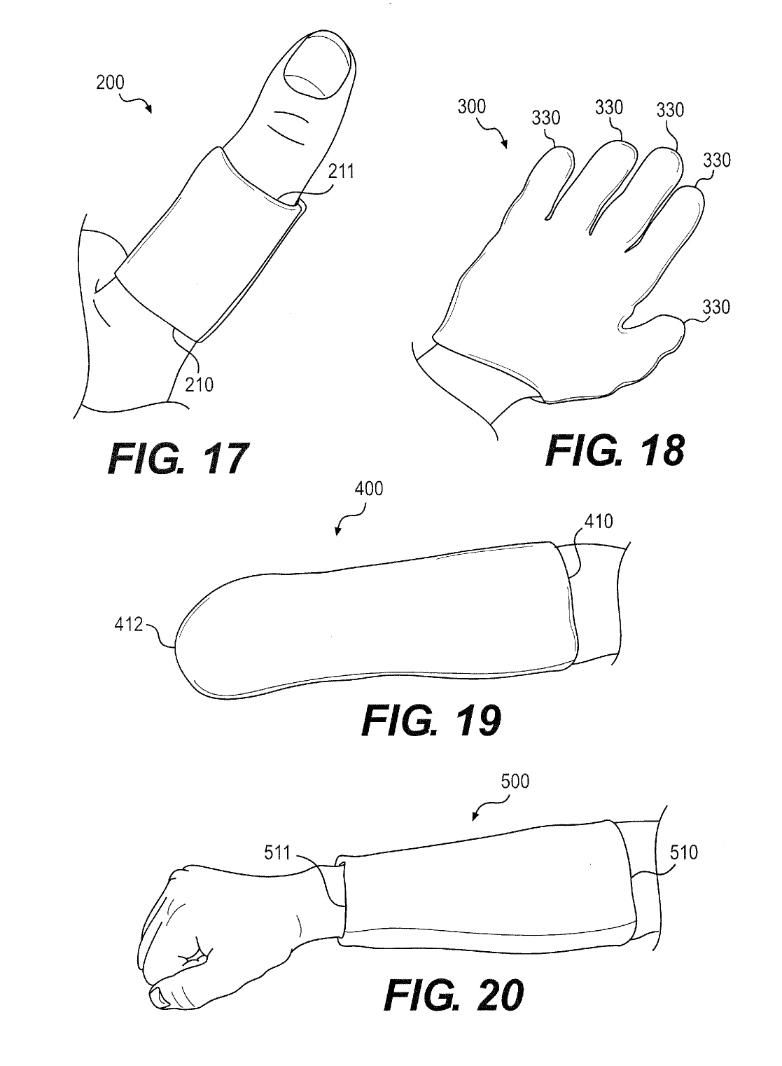

As noted herein, the wound dressing can be used on any suitable portion of the body. FIGS. 17-22 show examples of additional suitable uses for the wound dressing. Turning to FIG. 17, the wound dressing 200 can be formed as a ring with two open ends 210, 211 for covering a wound on an extremity such as a finger, toe, or penis. Alternatively, if the wound dressing is provided with an open end and a closed end, such as shown in FIG. 1, the closed end can be cut off to form the ring of FIG. 17.

As shown in FIG. 18, the wound dressing 300 can be provided as a glove for covering a hand. The glove can have one or more individual sleeves for receiving one or more fingers. For example, as shown in FIG. 18, individual finger sleeves 330 can be provided for receiving each finger. As another example, in some embodiments, the glove could be formed as a mitten with a single pocket for receiving all fingers except the thumb. The glove can cover the entire hand including all fingers. However, in certain embodiments, the one or more finger sleeves 330 can be trimmed from the glove if only the palmar and/or dorsal portions of the hand, or only certain fingers, are wounded and in need of covering with the wound dressing. In other embodiments, the wound dressing can be provided without finger sleeves 330 for fingers or only selective finger sleeves 330 for certain individual or combinations of fingers. The wound dressing 300 can be provided with the foam portions disposed on the interior such that the extremity can be inserted into the wound dressing 300. In other embodiments, the wound dressing 300 can be provided with the at least partially rolled over itself with at least a portion of the foam portions disposed on the exterior. In such embodiments, the wound dressing 300 can be rolled over itself as described above such that the wound dressing 300 does not have to slide over the wound for application.

Referring to FIG. 19, the wound dressing 400 can be provided in suitable sizes to receive and enclose wounds on limbs such as arms and legs. For example, the wound dressing can be provided with an open end 410 and a closed end 412 to completely enclose an end of a limb. Such a wound dressing can, for example, be suitable for use following amputation of a portion of an arm or leg.

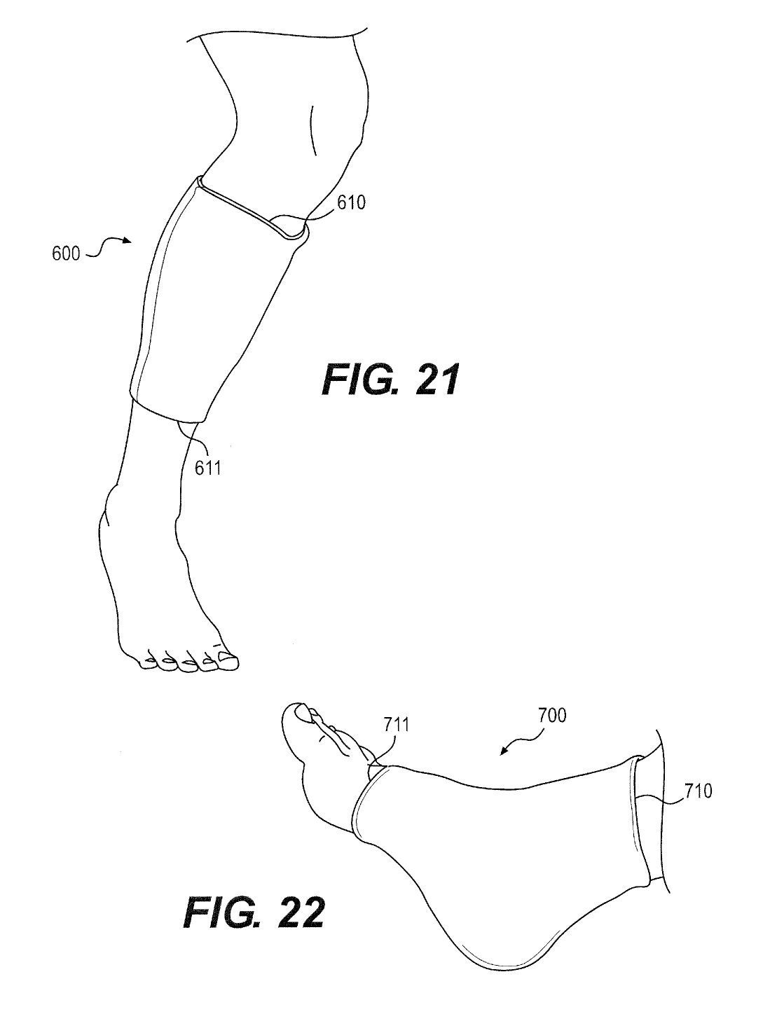

As shown in FIGS. 20 and 21, the wound dressing 500, 600 can have two open ends 510, 511, 610, 611 such that the wound dressing 500, 600 can be positioned over a portion of a limb, such as a forearm, calf, or any other suitable portion of a limb, while still permitting usage of an uncovered portion of the limb, such as a hand or foot. As another example, as shown in FIG. 22, the wound dressing 700 can have two open ends 710, 711 and be positioned over a joint, such as an ankle, knee, wrist, or elbow, to enclose and facilitate healing of a wound at or near the respective joint. As shown, the wound dressing 700 can be provided as a sock for covering the entirety or a portion of a foot. In certain embodiments, the wound dressing 700 can be a sock with a closed end for enclosing one or more toes in addition to at least a portion of a foot.

The foam forming the body can be a hydrophilic polymer-based foam of the type typically used for wound dressings. In this regard, polyurethane foams may be particularly desirable. By way of example only, and not limitation, potentially desirable polyurethane foams and methods of preparing such foams are described in U.S. Pat. Nos. 5,064,653 and 5,916,928 both to Sessions et al. the teachings of which are incorporated by reference as if fully set forth herein. Optionally, the foam may incorporate an anti-microbial agent. Exemplary anti-microbial agents include silver metal, silver alloys and silver salts such as silver nitrate. In this regard, the inclusion of silver metal in an amount of about 0.1% to about 2% by weight based on of the foam may be particularly preferred for some applications.

According to certain embodiments, the foam is formed from the reaction of water with isocyanate-capped polyurethane prepolymers as will be known to those of skill in the art. The amount of prepolymer in the reactant composition used to prepare the hydrophilic foam composition typically depends on its isocyanate functionality and the degree of crosslinking desired in the final foam product. In general, the greater the isocyanate functionality, the greater the degree of cross-linking in the cured foam product. Typically, the reactant composition will include from about 20% to about 60% by weight prepolymer. Preferably the reactant composition will include from about 45% to about 50% by weight of the prepolymer.

The reactant composition forming the foam may, if desired, further include a hydrophilic agent which is incorporated into the foam composition to enhance absorption of external liquid, such as wound exudate, and to retain such liquid in the foam composition. The hydrophilic agent incorporated into the foam composition is believed to absorb fluid from the wound to assist thickening of the blood, i.e., it serves as a hemostat. Absorption of exudate by the hydrophilic agent, and the subsequent swelling of the agent results in the removal of inflammatory exudates and particles that would otherwise hinder tissue repair or cause eschar formation. Necrotic debris and bacteria are likewise removed as autolysis, i.e. chemical debridement, is stimulated. Suitable superabsorbent polymers include sodium and aluminum salts of starch, grafted copolymers of acrylates and acrylamides, and combinations thereof, as well as polyacrylate salts. Of course, other absorbent materials may be used in combination with such highly absorbent polymers. When such agents are employed, either alone or in combination, the resulting foam composition desirably has the ability to hold at least about three times its weight in liquid. In the preferred embodiment, the resulting foam composition will have the ability to tightly hold at least about three times its weight in fluid. As used herein “tightly held” or “tightly bound” liquid means the relative amount of liquid retained by the sample after compression.

The amount of hydrophilic agent used and the type of agent, in terms of its fluid uptake, that may be satisfactorily used to make the foam composition is not critical, but is, instead, dependent on the intended application of the resulting foam composition. However, the amount of hydrophilic agent utilized should not be so great as to undesirably reduce the strength of the foam composition or result in a loss of polymer from the foam, although some loss of hydrophilic agent may be tolerated without adversely affecting the ability of the foam to absorb external liquids. The amount of hydrophilic agent employed in the reactant composition will also depend on the absorbency of the material used. As previously indicated, it is preferable that a sufficient amount of hydrophilic agent be employed so that the resulting foam composition is capable of absorbing at least about three times its weight in external liquid. Typically this can be achieved by including from about 5 wt. % to about 20 wt. % hydrophilic agent in the reactant composition.

The reactant composition of this invention may further include an adjuvant; preferably, a water-soluble adjuvant. The adjuvant is releasably carried by the resulting foam composition for subsequent release to a chosen situs of application. Release of the adjuvant occurs in the presence of an external liquid, such as wound exudate, which is preferentially absorbed by the foam composition. Absorption of the external liquid causes at least a portion of the adjuvant to be released.

It will be appreciated by those skilled in the art that not all of the liquid adjuvant is necessarily released (or need it be) in the presence of the external fluid. However, a sufficient amount of adjuvant must be released in order to achieve the desired result. To that end, it will be appreciated that the efficacy of the adjuvant is realized upon its release from the foam composition to the wound cavity.

Prior to curing, the adjuvant serves as a plasticizer for the reactant composition. It extends the curing time of the composition thereby allowing it to be more thoroughly mixed and formed. Once cured, the foam composition is softened by the adjuvant, allowing the foam to be more pliable and more easily applied to the skin surface or other surface of choice. Additionally, the adjuvant may be somewhat hygroscopic lending further to the hydrophilic nature of the foam composition.

Adjuvants suitable for use in the foam composition of the present invention are mono-, di- and polyhydric alcohols. Preferably the adjuvants are water soluble so that they may be readily released from the composition upon contact of the foam composition with an external liquid. It is also preferred that the adjuvant be compatible with therapeutic or other agents which may be carried by the adjuvant for subsequent delivery to the situs of application. Suitable adjuvants include water soluble alcohols, including monols, diols and polyhydric alcohols. Examples of monols include ethyl alcohol and isopropyl alcohol. Exemplary of suitable diols are propylene glycol, polyethylene glycol, and polypropylene glycol. Exemplary of suitable polyhydric alcohols are glycerin, 1,2,4-butanetriol, trimethylolpropane, pentaerythritol, and sorbitol. In general, the molecular weight of the alcohols should be less than about 1000. Mixtures of alcohols can likewise be used. Glycerin may be a particularly preferred adjuvant because it has the attributes of a medicament, cosmetic, or therapeutic agent. Various additional medicaments, cosmetics, and therapeutic agents may, if desired, be carried with the adjuvant and released with it to the desired situs. This release thus allows the transmission of such therapeutic or other agents carried in the adjuvant to the area of application outside the foam composition, further assisting in the beneficial treatment of the wound.

The amount of adjuvant included in the reactant composition should preferably be sufficient to impart softness and pliability to the foam composition and be capable of delivering a therapeutic agent or the like, if included, to the environment of application. However, the volume of adjuvant should not be so great as to weaken or gel the composition. Generally, it has been found that the amount of adjuvant in the reactant composition should be from about 5 wt. % to about 30 wt. % of the reactant composition.

A wetting agent optionally may be included in the reactant composition to provide more uniform wetability of the resulting foam. The wetting agent also aids in controlling the cell size of the foam and in the reticulation of the final foam. Wetting agents suitable for use include non-ionic surfactants. Examples of materials that may be used as the wetting agent, either alone or in admixture, include block copolymers of ethylene oxide and propylene oxide, ethoxylated sorbitan fatty acid esters, glycerol esters, polyglycerol esters, and silicone fluids as will be well known to those of skill in the art. Generally, the amount of wetting agent should be from about 1% to about 10% by weight of the reactant composition, preferably from about 5% to about 7% by weight. The wetting agent should not react with the foam composition or any component of the foam formulation to create difficulties during foam formation or to adversely affect the desired characteristics of the foam composition in use or while being stored.

As will be appreciated by those of skill in the art, water is used in the initiation of the foaming reaction. It should be appreciated that the source of the water for the foaming reaction is not critical. The water so required may be provided as a separate component of the reactant composition, or, for example, it may be provided by one of the other components of the reactant composition. By way of illustration, and not in limitation, the required water may be provided with an aqueous-based cosmetic which may be incorporated into the foam composition. The type of water used is likewise not critical. However, for medical applications, purified water such as deionized or distilled water may be used. Saline solutions may also be used satisfactorily.

It will be appreciated that the relative proportion of prepolymer, adjuvant and hydrophilic agent, if the latter two are included in the reactant composition, can be varied over wide ranges in order to prepare a hydrophilic foam composition having the desired release and exchange characteristics previously described, while likewise providing a foam composition that is aesthetically satisfactory, insofar as its oilyness, touch, appearance and general feel.

As will be appreciated, while a potentially desirable foam product may be manufactured using an isocyanate-capped prepolymer, such prepolymers are exemplary only. Accordingly, it is contemplated that virtually any prepolymers yielding foams suitable for introduction into a human body may be satisfactorily employed.

In order to form the layers of the wound dressing, the raw material constituents of the foam such as adjuvant (or organic phase), prepolymer, and aqueous phase are transferred via inlet tubes to a suitable reaction vessel for combination and reaction. The reaction vessel merely serves to mix the reactants sufficiently such that they will react to form the reaction product. The reaction vessel is preferably equipped with speed-controllable mixing paddles to blend the phases and a temperature control means for controlling the temperature of the reactants.

The mixing speed of the vessel and its temperature are preferably set to a predetermined level as a variance in either parameter will affect the properties of the resulting foam. Generally, the predetermined levels are dependent on the flow rates of each component and more specifically on the combined flow rate. For example, if the mixer revolutions per minute (rpm) is too low, inadequate mixing of the reactants results. If the mixer rpm is too high, the heat build up due to the high setting increases the reaction rate of the reactants, thereby effecting the subsequent processing of the reaction product.

The temperature of the mixer is generally kept lower than the temperature of the reactants because the reaction itself is exothermic. If the temperature is too high, the reaction will proceed at a much higher rate, thereby effecting subsequent processing of the foam, and also shortening the cure time. Excessive temperatures can also cause an imbalance in carbon dioxide generation and polymerization which may result in a nonuniform product.

After the mixing process is completed, the reaction product may be discharged from the vessel through a nozzle. During the discharge of the reaction product, the urethane film may be passed onto a continuous conveyor traveling below the nozzle. The reaction product from the vessel is deposited on top of the film.

The flow rate of the reaction product from the nozzle and the velocity of the film are set to control the thickness and width of the resulting foam sheet. In this regard, the velocity of the film is directly proportional to the reaction time of the reaction product prior to compression by a first set of compression rollers and affects the nature of the reaction product.

By way of example only, the conveyer may be designed so as to allow the velocity of the film to vary from about 0.1 to about 11 feet per minute, with the rate at which the reaction product is deposited through nozzle being within the range from about 0.1 to about 2.0 pounds per minute. According to one potentially desirable process where the reaction product leaves the reaction vessel at 90 degrees Fahrenheit after being mixed at 2500 rpm, the reaction product is deposited at a rate of approximately 0.7 pounds per minute and the film travels at a velocity of about 4 feet per minute.

After the reaction product is deposited from nozzle, but before the reaction product is subjected to its initial compression by rollers, an overlying cover layer of substantially impermeable character is applied. The cover layer is preferably release coated, and is thus releasably adhered to the product. One potentially desirable cover layer that may be used is a silicone coated polystyrene sheet. However, any substantially impermeable material that may be easily removed from the final formed foam may be used.

Subsequent to, or simultaneously with, the introduction of the cover layer, the formed composite is subjected to a compressive force which serves to control the thickness of the resulting foam sheet product. According to a potentially desirable practice, the first compression may take place as the reaction product takes on a cream state and begins to foam and rise. While it is contemplated that the composite may undergo only one compression, it is preferred that it undergo multiple compressions. The compressions are preferably accomplished on a continuous basis by passing the composite through a series of compression rollers, each of which defines a gap therebetween. The compression rollers compress and spread the creamed foam so as to effect a reduction in foam thickness of from about 5 to about 95 percent of the foam thickness just prior to compression. Reductions of that magnitude may be effected for each of a plurality of compressions. It will be appreciated by those skilled in the art that the number of compressions, degree of compression, and the timing of the compressions may be adjusted to achieve a desired final foam character. More specifically, the density, thickness, width, and appearance of the product will be affected. In order to determine the number and degree of compressions for the particular reaction product and processing conditions employed, a measurement of the foam thickness of the reactant product that has been removed from the conveyor just after each sequential compression may be taken after the foam has been allowed to rise to its fullest extent. This measurement may then be compared with a measurement taken of the thickness of the foam reaction product that has similarly been allowed to rise to its fullest extent without undergoing that compression. Such a comparison will allow an operator to determine both the number of compressions and the degree of compression needed to attain a foam having the desired final thickness.

In certain embodiments, the initial compression preferably reduces the thickness of the reaction product by about 80 percent, and each subsequent compression reduces the thickness by about 40 percent. Compressing the composite in this manner results in a superior final foam product that will emerge having a specific, predetermined thickness. By way of example only, when the velocity of the film is about 5 feet per minute, and the reaction product is deposited at a rate of 0.2 pounds per minute, it is preferred that the initial compression takes place within about 2 seconds after the reaction product leaves the nozzle. The second and third compressions, each of which compresses the foam about 40 percent, should then occur within 55 and 70 seconds, respectively, after the material has left the nozzle.

In accordance with a potentially desirable practice, final curing of the foam takes place at ambient conditions following compression without the introduction of heat. If desired, the composite exiting the final compression rolls may subsequently be subjected to drying means wherein moisture level within the foam is reduced to a predetermined level. Preferably the moisture level in the final foam product is about 10 percent or less by weight. Advantageously, drying is carried out using a hot air impingement dryer, with the air that is used for the dryer being first drawn through a particulate filter. According to one potentially desirable practice, the drying temperature is in the range from about 100 degrees Fahrenheit to about 175 degrees Fahrenheit and most preferably about 140 degrees Fahrenheit.

Following drying, the resultant composite may be collected in a jelly-roll arrangement about the take-up winder. Thereafter, sections of the resultant composite may be removed from the collection roll and cut to predetermined lengths and shapes as described. The cover layer may be removed before such cutting. Virtually any three-dimensional shape may be achieved by folding the composite and adjoining edges together by adhesives, laser butt welding or other suitable techniques as may be know to those of skill in the art. The resultant structure is then useable as a wound dressing providing absorption and high wicking capacity of wound fluids.

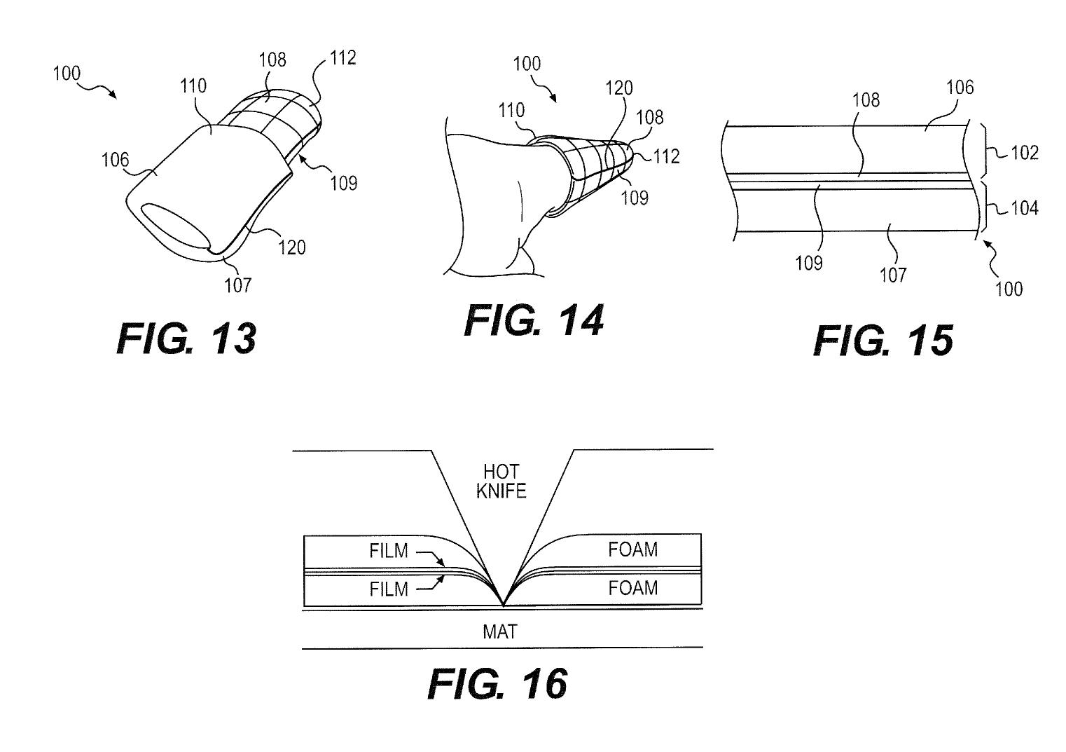

Turning to FIG. 16, the wound dressing can be formed with an automated process. Two sheets of the material forming the layers of the wound dressing can be placed on top of each other on a heat-resistant mat. The layers can be disposed with the film portions of the layers in contact with one another. A temperature-controlled hot knife can be pressed at a controlled pressure against both sheets into the mat for a controlled time. The knife cuts the layers and welds the film portions together to faun a seam 120 as shown in FIGS. 13 and 14. In some embodiments, the knife surface temperature can be approximately 160-170° C., and the dwell time can be approximately 5-7 seconds. The seam can be sized and/or positioned to minimize contact between the seam and the extremity, and thus minimize the contact between the seam and the wound. The wound dressing can be cut into any suitable shape.

In some embodiments, instead of using two sheets of material placed on top of one another in order to form the wound dressing, it will be appreciated that a single sheet of material can be folded over itself For example, the single sheet can have a foam portion and a film portion. The single sheet can be folded over itself with the film portion disposed between the foam portion. A temperature-controlled hot knife can be pressed at a controlled pressure against the folded sheet into the mat for a controlled time. The knife cuts the sheet to form the layers and welds the film portions together to foam a seam.

In further embodiments, the cutting and joining process can be conducted separately. For example, two separate pieces of a desired shape and size can be cut from one or more sheets. The film portions of each piece can be disposed in contact with another and between the foam portions. To form the wound dressing, the pieces can be aligned and joined along a plurality of the edges by heat welding or other suitable process discussed herein. In other embodiments, a single piece may be cut from a sheet with a length sufficient to fold over itself The film portion can be disposed between the foam portion. To form the wound dressing, the edges of the single piece can be aligned and joined along a plurality of the edges by heat welding or other suitable process discussed herein.

It will be appreciated that the wound dressing can be formed in any suitable manner. Silicone lubricant can be added to the film portions of the first and second layers to ease the rolling process of the wound dressing onto an extremity in those embodiments where rolling the wound dressing over itself is desirable.

It will be appreciated that the wound dressing can be any suitable shape and size to at least partially surround any suitable body part. In addition, the wound dressing can be provided in any suitable shape and size and be trimmed as needed for customization in covering a particular wound. The wound dressing can be applied in any suitable manner, such as sliding over the extremity or rolling over itself to cover the extremity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A wound dressing for an extremity comprising:
a first layer including a perimeter and a polyurethane foam disposed on a film; and
a second layer including a perimeter and a polyurethane foam disposed on a film, the second layer being joined to the first layer around at least a portion of the perimeters of the first layer and the second layer to form a space between the first layer and the second layer and to form a first open end.

2. The wound dressing of claim 1 further comprising a closed end opposing the first open end.

3. The wound dressing of claim 2 wherein the closed end is curved.

4. The wound dressing of claim 2 wherein the closed end is folded into the space between the first layer and the second layer to form a dimple.

5. The wound dressing of claim 4 further comprising a dressing insert removeably disposed in the dimple.

6. The wound dressing of claim 5 wherein the dressing insert has a tab.

7. The wound dressing of claim 1 wherein the first layer film and the second layer film form an exterior of the wound dressing.

8. The wound dressing of claim 7 wherein the first layer foam and the second layer foam are disposed between the first layer film and the second layer film.

9. The wound dressing of claim 1 further comprising a second open end opposing the first open end.

10. The wound dressing of claim 1 wherein the wound dressing is shaped as a glove.

11. The wound dressing of claim 1 wherein the wound dressing is shaped as a ring.

12. The wound dressing of claim 1 wherein the wound dressing is shaped as a sleeve.

13. The wound dressing of claim 1 wherein at least one of the first layer foam and the second layer foam include an anti-microbial agent.

14. The wound dressing of claim 1 wherein at least one of the first layer foam and the second layer foam include a hydrophilic agent.

15. The wound dressing of claim 1 wherein at least one of the first layer foam and the second layer foam include an adjuvant.

16. The wound dressing of claim 1 wherein the first layer film and the second layer film are joined by a heat welded seam.

17. The wound dressing of claim 1 wherein at least one of the first layer film and the second layer film include an indicia representing an exterior of the wound dressing.

18. A method of using a wound dressing for an extremity comprising:
providing a wound dressing, the wound dressing including,
a first layer including a perimeter and a polyurethane foam disposed on a film; and
a second layer including a perimeter and a polyurethane foam disposed on a film, the second layer being joined to the first layer around at least a portion of the perimeters of the first layer and the second layer to form a space between the first layer and the second layer and to form an open end, the joined first layer and second layer including a closed end opposing the open end, the closed end being folded into the space between the first layer and the second layer to form a dimple;
inserting a tip of an extremity into the dimple; and
rolling the wound dressing over the extremity to enclose at least a portion of the extremity.

19. The method of claim 18 further comprising removing a dressing insert from the dimple.

20. The method of claim 18 wherein the extremity is selected from the group consisting of a finger, a toe, a hand, a foot, an arm, a leg, and a penis.
